(19) United States
(12) Patent Application Publication
(10) Pub. No.: US 2013/0158424 A1
(43) Pub. Date: Jun. 20, 2013

(54) METHOD AND SYSTEM FOR DETECTING RESPIRATORY INFORMATION

(71) Applicants: Robert Pinter, Lubeck (DE); Gerhard Spekowius, Eindhoven (NL); Donghai Yu, Shanghai (CN); Harald Reiter, Eindhoven (NL); Andreas Schupp, Hong Kong (CN)

(72) Inventors: Robert Pinter, Lubeck (DE); Gerhard Spekowius, Eindhoven (NL); Donghai Yu, Shanghai (CN); Harald Reiter, Eindhoven (NL); Andreas Schupp, Hong Kong (CN)

(73) Assignee: KONINKLIJKE PHILIPS ELECTRONICS N.V., EINDHOVEN (NL)

(21) Appl. No.: 13/392,907

(22) PCT Filed: Aug. 31, 2010
(86) PCT No.: PCT/IB10/53894

§ 371 Date: Feb. 28, 2012

(30) Foreign Application Priority Data
Aug. 31, 2009 (CN) 200910170970.5

(51) Int. Cl. A61B 5/08; A61B 5/00

(52) U.S. Cl. CPC A61B 5/08; A61B 5/7278; A61B 5/742; A61B 5/7405; A61B 5/7475
USPC 600/529

(57) ABSTRACT

The invention proposes a method of detecting respiratory information of a user. The method comprises a step of instructing the user to perform on a device a movement reflecting his respiratory action; a step of generating by said device, a signal reflecting said movement; and a step of deriving the user's respiratory information from said signal. By using this method, the respiratory information is easily detected without attaching any sensor on the user's chest.

### FIELD OF THE INVENTION

This invention relates to a method and system of detecting respiratory information.

### BACKGROUND OF THE INVENTION

Respiratory exercises are an effective relaxation technique and are also good for the treatment of high blood pressure. The known device for respiratory exercises teaches a respiratory technique with the help of measurements and feedback to the user. Ideally, the respiratory action of the user is measured, and then compared with previously defined requirements, and instructions are given such that the user is guided towards a respiratory technique according to the predefined requirements.

However, measuring respiratory action requires attaching some kind of sensor to the chest, for example, a chest belt with an integrated sensor to be worn during the exercise, which is undesirable and uncomfortable.

In the past, people accepted devices that came with cables attached to their body or even as an add-on box connected to their computer. But today, the need for wearable solutions that do not require a complicated installation of sensors and wires is overwhelming. Ideally, the device should be ready for operation as soon as the user takes it in his hands.

### SUMMARY OF THE INVENTION

It is therefore an object of this application to provide a method and a device for detecting the respiratory information of a user in a comfortable way.

In accordance with one aspect, a method of detecting respiratory information of a user is proposed. The method comprises the steps of:
instructing the user to perform on a device a movement reflecting his respiratory action;
generating, by said device, a signal reflecting said movement;
deriving the user's respiratory frequency from said signal.

According to an embodiment, the method may further comprise a step of selecting a part of the signal having periodical variation; wherein the deriving step derives the user's respiratory information from said part of the signal.

In accordance with another aspect, a system for detecting respiratory information of a user is proposed. The system comprises:
a first unit for instructing the user to perform on a device a movement reflecting his respiratory action;
a device being adapted to generate a signal reflecting said movement;
a second unit for deriving the user's respiratory information from said signal.

According to an embodiment, the system may further comprise a unit for selecting a part of the signal having periodical variation; wherein the user's respiratory information is derived from said part of the signal.

Furthermore, the first unit can be used to provide a breathing instruction to the user.

The invention is in particular useful for handheld devices that need to determine the user's respiratory information. By using this method, measuring the respiratory action of a user without attaching a sensor to the user's body is realized. Furthermore, the user's breathing exercises are improved by emphasizing the exhalation phases through the movement performed on the device which the user has to perform according to this invention. The system is easy to operate and robust.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference Figures quoted below refer to the attached drawings.

### BRIEF DESCRIPTION OF THE DRAWINGS

FIG. 1 depicts a system for detecting the respiratory information of a user according to an embodiment of the invention;

FIG. 2 depicts a flow chart of the detection of respiratory information according to an embodiment of this invention;

FIG. 3 depicts an embodiment of the device 130.

Throughout the above drawings, like reference numerals will be understood to refer to like, similar or corresponding features or functions.

### DETAILED DESCRIPTION OF THE INVENTION

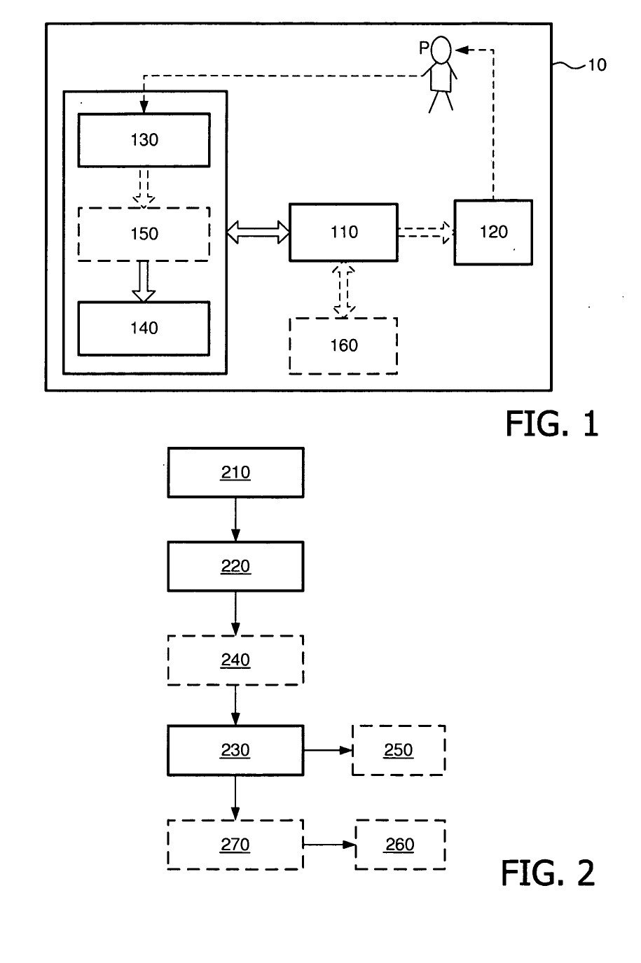

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes. In the block diagram of FIG. 1, the dashed portion shows the element that may be removed from the system in various embodiments; in the flowchart of FIG. 2, the dashed portion shows the step that may be removed from the process in various embodiments. Where an indefinite or definite article is used when referring to a singular noun, e.g. “a”, “an”, “the”, this includes a plural of that noun unless something else is specifically stated.

FIG. 1 depicts by way of example a system 10 for detecting the respiratory information of a user P and FIG. 2 depicts the flow chart of the detection of the respiratory information according to an embodiment of this invention. System 10 includes a first unit 120 and a device 130 and a controller 110.

The first unit 120 is used for instructing the user P to perform on the device 130 a movement reflecting his respiratory action (step 210). The first unit 120 may comprise for example a video display device having a display screen. It may also comprise an audio playing device having a speaker (not shown in the Figures). In this case, the instructing step 210 can be performed for example by displaying an instruction on the display screen or by playing the instruction via the speaker. The instruction for example can be a demo showing how to perform the movement correctly. Alternatively, the instruction can be an audio signal.

Alternatively, the instructing step 210 may also be performed by printing the instruction in the format of text on the surface of the device 130 so as to remind the user to perform the movement that will follow his respiratory frequency. In this case, the surface of the device 130 on which the instruction is printed is the first unit 120.

Alternatively, the instructing step 210 may also be performed by printing the instruction on the user manual or leaflet or any type of advertisement relating to the device 130. The user manual normally is packaged together with the system 10 when the system 10 is sold. In this case, the first unit 120 carries the user manual or leaflet or advertisement.

The movement performed by the user in this invention can be controlled completely by the user's consciousness, e.g. the movement controlled by the user's hand. By contrast, the user's chest movement resulting from the user's respiratory action is not completely controllable by the user's consciousness and therefore is not the movement of this invention. Depending on different types of device 130, the movement can be different, which will be explained in detail herein below.

The device 130 is controlled by the controller 110 to generate a signal reflecting the movement performed by the user on the device 130 (step 220).

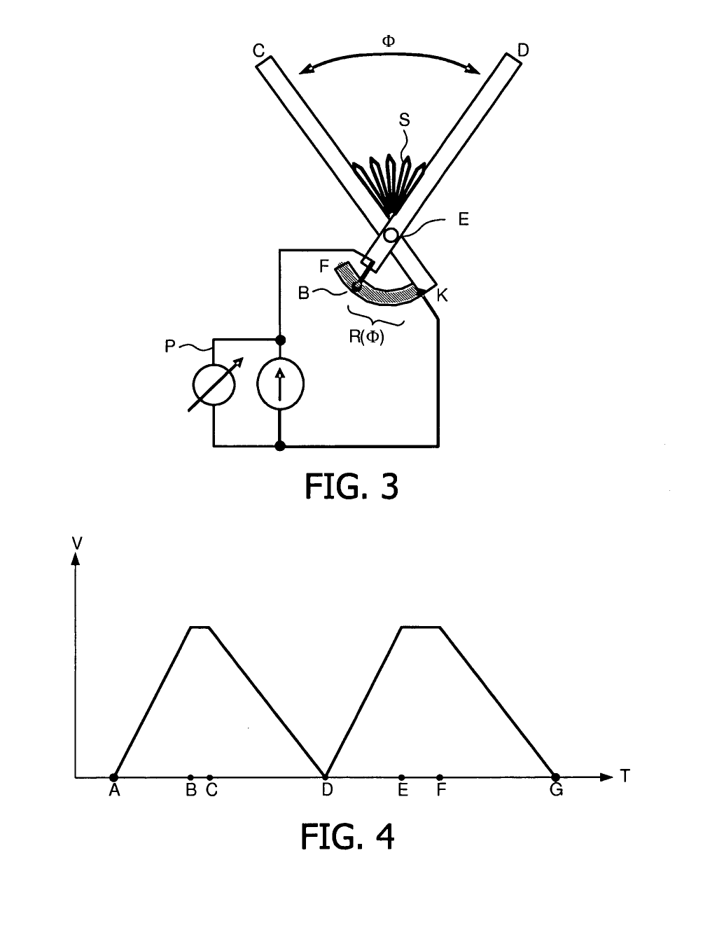

FIG. 3 depicts a first embodiment of the device 130. In this embodiment, the device 130 comprises a mechanical unit having a movable electric contact (B) and a fixed electric contact (K) and a circuit for measuring the resistance between the two contacts. The mechanical unit is made of two rods CK and BD that are connected at the joint E. Spring S installed at the joint E forces the free ends of the rods apart, so that the mechanical unit assumes the shape of a “V”. An extension part KF is made of a resistive material and fixed to KC at point K (fixed electric contact). The extension part KF is designed in such a way that the free end B (movable electric contact) of rod BD can slide along KF. The circuit could be for example a potentiometer P to measure the voltage between the two contacts. Since U=I*R(Φ), the resistance change is linear to the voltage change which can be measured by the potentiometer P.

In this embodiment, the signal generated by the device 130 is based on the voltage measurement result.

The movement instructed by the first unit 120 may be for example: to press the free ends of the rods together, working against the spring force during exhalation, and release them during inhalation. In this way, the user's respiratory information is reflected by the movement performed by the user on the mechanical unit. And this can be monitored by measuring the voltage between point B and point K.

In this way, respiratory action is measured without a sensor on the subject's chest.

In another embodiment, the mechanical unit in the first embodiment is replaced by a deformable object and the potentiometer is replaced by a sensor for detecting the deformation of the object. The deformation is caused by the movement performed by the user, and the signal is generated according to the deformation detection result. The deformable object could be for example: a rubbery ball, or an extendible plastic strap. The sensor for detecting the deformation of the object could be for example: an inner ball (air) pressure sensor, digital holographic interferometry, resistive motion position sensors, magnetostrictive sensors, linear wire potentiometer/encoder. The movement could be to extend/release or compress/release the object following the user's respiratory action.

In yet another embodiment, the mechanical unit in the first embodiment is replaced by a touch pad and the potentiometer is replaced by a pressure sensor, for example a piezoelectric sensor, for detecting the pressure acting on the touch pad. In this embodiment, the movement performed by the user should be to press/release the touch pad that follows the user's respiratory action. And then a signal is generated according to the detection result of the pressure sensor. As a result, the signal reflects the user's respiratory action.

In a further embodiment, the device 130 comprises an object having a movable part and a motion sensor attached to the movable part for detecting the movement of said movable part. The motion sensor could be for example an accelerometer. In this case, the movement performed by the user should cause the movable part to move forwards and backwards following his respiratory action. And thus the signal generated by the device 130 reflects the movement and therefore reflects the user's respiratory action.

Consequently, the signal is generated by the device 130 according to the detection result of a different type of sensor in a different embodiment. For example, in the first embodiment, the signal may be generated by directly recording the measured resistance over the relevant period of time. The signal could be, for example, the voltage of BK over the relevant period of time; the pressure of the touch pad over the relevant period of time; the deformation of the object over the relevant period of time.

The system 10 also includes a second unit 140 for deriving the user's respiratory information from the generated signal (step 230).

A typical breathing pattern of a human being can be separated into four parts: the inhaling, the transition phase between inhaling and exhaling, the exhaling and the pause between each breathing cycle. Each of these parts is highly subjective and differs from one person to the other. The respiratory information could be derived by analyzing the signal. For instance, the length of inhale phase and exhale phase and the transition phase, the pause between each breathing cycle and the breathing frequency can be extracted.

For example, according to the first embodiment, FIG. 4 shows that the signal reflects the voltage change over the relevant period of time. From the signal shown in FIG. 4, in one breathing cycle AD, if the duration AB is the inhalation phase, then the duration BC is the transition phase, and duration CD is the exhalation phase. Also, the breathing frequency can be derived by calculating the time interval between each vicinal maximum value and minimum value, for example, the duration of AD or the average of the duration AD and DG could be used to calculate the breathing frequency.

To determine which phase is an inhale phase depends on whether the breathing instruction is provided to the user synchronously. If there is a breathing instruction, according to the instruction, the inhale phase and exhale phase can be determined from the generated signal.

In a similar way, the respiratory information can be derived according to other types of signal, e.g. the deformation of the object, etc.

In an embodiment, if the signal generated by the device 130 includes noise, system 10 may further comprise a third unit 150 for selecting part of the signal having periodical variation (step 240). The noise is induced because for example the user stops performing the instructed movement or performs the movement without following his respiratory action. In this case, the unit 150 can select the part of the signal having the periodical variation and this part is used to derive the user's respiratory information. More precisely, the variation period is compared with a predefined breathing requirement, for example the breathing frequency range. If the variation period of a part of the signal is beyond the breathing frequency range, this part is deemed to be noise and thus will not be selected in the selecting step 240.

In another embodiment, the above mentioned video display device or the audio playing device is further used for providing the derived respiratory information to the user (step 250). The derived respiratory information may also be stored in the memory for the user to check later.

In another embodiment, the above mentioned video display device or the audio playing device is further used for providing a breathing instruction to the user based on a predefined breathing requirement (step 260). In this case, the system 10 is not only used for detecting the user's respiratory information but also for instructing the user to breath in a healthy way; for example, it is known that it is good to breath out (exhale phase) longer than to breath in (inhale phase); a predefined breathing style could be: 4 seconds to breath in and 6 seconds to breath out.

In another embodiment, when there is a predefined breathing requirement, the system 10 may further comprise a fourth unit 160 for comparing the derived respiratory information with the predefined breathing requirement (step 270).

Furthermore, the breathing instruction may be updated and provided based further on the comparison result. For example, if the comparison result shows that the user's actual breathing frequency is very different from the breathing instruction, the system could lower the requirement so that it can be easily met by the user or the system may generate an alarm signal to remind the user that he should try to follow the breathing instruction.

There are numerous ways of implementing functions by means of items of hardware or software, or a combination of hardware and software. In this respect, the drawings are illustrative, each representing only one possible embodiment of the invention. For example, the above mentioned controller 110 can be implemented by a memory with instruction data, or by a microchip, and so can unit 120, unit 140, unit 150, unit 160; wherein the different functions can also be implemented by a memory with different instruction data.

It should be noted that the above described embodiments are given for describing rather than limiting the invention, and it is to be understood that modifications and variations may be resorted to without departing from the spirit and scope of the invention as those skilled in the art readily understand. Such modifications and variations are considered to be within the scope of the invention and the appended claims. The protective scope of the invention is defined by the accompanying claims. In addition, the reference numerals in the claims should not be interpreted as a limitation to the claims.

1. A method of detecting respiratory information of a user, comprising the steps of:
instructing the user to perform on a device a movement reflecting his respiratory action;
generating, by said device, a signal reflecting said movement;
deriving the user's respiratory information from said signal.

2. A method according to claim 1, said method further comprising a step of selecting part of the signal having periodical variation;
wherein said deriving step derives the user's respiratory information from said part of the signal.

3. A method according to claim 2, wherein the variation period of said part of the signal is within a predefined range.

4. A method according to claim 1, said method further comprising a step of providing said derived respiratory information to the user.

5. A method according to claim 1, said method further comprising a step of providing a breathing instruction to the user according to a predefined requirement.

6. A method according to claim 5, said method further comprising the steps of:
comparing said derived respiratory information with said predefined requirement;
wherein said step of providing a breathing instruction is further based on the result of said comparing step.

7. A system for detecting respiratory information of a user, comprising:
a first unit for instructing the user to perform on a device a movement reflecting his respiratory action;
a device being adapted to generate a signal reflecting said movement;
a second unit for deriving the user's respiratory information from said signal.

8. A system according to claim 7, said system further comprising a third unit for selecting part of said signal having periodical variation; wherein said user's respiratory information is derived from said part of the signal.

9. A system according to claim 7, wherein said first unit comprises at least one of the following devices:
a video display device;
an audio playing device.

10. A system according to claim 7, said device comprising a deformable object and a sensor for detecting the deformation of the object; wherein the deformation is caused by said movements, and said signals are generated according to the deformation detection result.

11. A system according to claim 7, said device comprising an object having a movable electric contact and a fixed electric contact and a potentiometer for measuring the voltage between the two contacts; wherein said movable electric contact is moved by said movements, and said signals are generated according to the voltage measurement result.

12. A system according to claim 7, said device comprising an object having a touch pad attached to it with a pressure sensor for detecting the pressure acting on the touch pad; wherein said pressure is caused by said movements, and said signals are generated according to the detection result of the pressure sensor.

13. A system according to claim 7, said device comprising an object having a movable part and a motion sensor attached to said movable part for detecting the movement of said movable part; wherein the movement of said movable part is caused by said movement of the user, and said signals are generated according to the detection result of the motion sensor.

14. A system according to claim 9, wherein said video display device and the audio playing device are further used for providing a breathing instruction to the user, based on a predefined requirement.

15. A system according to claim 14, further comprising a fourth unit for comparing said derived respiratory information with the predefined requirement; wherein the breathing instruction is provided based further on the comparison result.
